(19) United States
(12) Patent Application Publication
(10) Pub. No.: US 2024/0407685 A1
(43) Pub. Date: Dec. 12, 2024

(54) METHOD AND APPARATUS FOR SUPPORTING AUTISM SPECTRUM DISORDER DIAGNOSIS BASED ON ARTIFICIAL INTELLIGENCE

(71) Applicant: ELECTRONICS AND TELECOMMUNICATIONS RESEARCH INSTITUTE, Daejeon (KR)

(72) Inventors: Byung-Ok HAN, Daejeon (KR); Moon-Ki BACK, Daejeon (KR); Jang-Hee YOO, Daejeon (KR); Cheol-Hwan YOO, Daejeon (KR)

(21) Appl. No.: 18/623,224

(22) Filed: Apr. 1, 2024

(30) Foreign Application Priority Data
Jun. 12, 2023 (KR) 10-2023-0075037
Jan. 3, 2024 (KR) 10-2024-0000710

(51) Int. Cl. A61B 5/16; A61B 5/00; G06V 10/70; G06V 10/80; G06V 20/40; G06V 40/16; G06V 40/18; G06V 40/20; G10L 17/02; G10L 25/57; G10L 25/66; G16H 50/20

(52) U.S. Cl. CPC A61B 5/16; A61B 5/4803; G06V 10/768; G06V 10/806; G06V 20/46; G06V 40/171; G06V 40/193; G06V 40/20; G10L 17/02; G10L 25/57; G10L 25/66; G16H 50/20

(57) ABSTRACT

Disclosed herein is a method for supporting Autism Spectrum Disorder (ASD) diagnosis based Artificial Intelligence (AI). The method includes extracting a detection area and voice corresponding to an inspector from an input video, extracting a detection area and voice corresponding to an assessment subject from the input video, extracting a feature of the inspector and a feature of the assessment subject, and extracting an interaction feature using the feature of the inspector and the feature of the assessment subject.

### CROSS REFERENCE TO RELATED APPLICATIONS

This application claims the benefit of Korean Patent Applications No. 10-2023-0075037, filed Jun. 12, 2023, and No. 10-2024-0000710, filed Jan. 3, 2024, which are hereby incorporated by reference in their entireties into this application.

### BACKGROUND OF THE INVENTION

### 1. Technical Field

The present disclosure relates to Artificial Intelligence (AI) technology for supporting Autism Spectrum Disorder (ASD) diagnosis.

More particularly, the present disclosure relates to technology for supporting ASD diagnosis using interaction between an inspector and an assessment subject and context features.

### 2. Description of Related Art

Autism Spectrum Disorder (ASD) assessment is conducted using semi-structured diagnostic tools (autism diagnostic observation schedule (ADOS), etc.) in such a way that an inspector (expert) directly observes the communication, social interaction, play, and restricted and stereotyped behavior of an assessment subject and scores the observed behavior based on the evaluation criteria of the diagnostic tool. These ASD diagnostic tools require a long training process for the use thereof, exhibit inconsistency in diagnosis depending on the experience and ability of individual experts, and require at least six to seven hours of test time and a significant amount of resources for diagnosis of each child.

With the advancement of AI technology, automation of ASD screening/diagnosis has been continuously attempted. Conventional technologies have focused on eliciting nonverbal responses from a subject using structured video content capable of providing social stimuli and analyzing and diagnosing the social behavior of the subject using an automated AI algorithm. That is, the nonverbal responses of the subject are evaluated using technology for analyzing gaze, a facial expression, an action, and nonverbal vocalization based on AI, and the resultant information is used for an AI diagnostic.

For example, video content capable of eliciting praise or laughter from a subject is created, and while the subject is watching the video, the responsive smile of the subject is recognized by AI technology and used for ASD diagnosis. Although the behavior of a subject in response to a stimulus is one of key indicators in ASD diagnosis, relying solely on the subject's response behavior information has the following problems:
focusing solely on the response of a subject for observation and ASD diagnostic evaluation: It may be inappropriate to diagnose in such a way that AI technology focuses only on the response of a subject, which is one aspect of social interaction with others. That is, because it is necessary to organically analyze the social behavior of the subject according to the behavior of others, integrated analysis of the social interaction behavior of the subject and others is required.
observation and ASD diagnostic evaluation that are not based on context: One of key indicators in ASD diagnosis is whether a subject can communicate naturally based on context information (environmental/situational factors) in social interaction. The conventional AI technologies have focused only on action analysis based on the responses of a subject without considering context information, and it is difficult to analyze a subtle difference of social interaction behavior based on context.

### DOCUMENTS OF RELATED ART

(Patent Document 1) Korean Patent No. 10-1276097, titled “Multi-simulator and simulation method for treating ADHD”.

### SUMMARY OF THE INVENTION

An object of the present disclosure is to support Autism Spectrum Disorder (ASD) diagnosis by analyzing the interaction behavior between an inspector and an assessment subject.

Another object of the present disclosure is to support ASD diagnosis by analyzing social context information.

In order to accomplish the above objects, a method for supporting ASD diagnosis based on AI according to an embodiment of the present disclosure includes extracting a detection area and voice corresponding to an inspector from an input video, extracting a detection area and voice corresponding to an assessment subject from the input video, extracting a feature of the inspector and a feature of the assessment subject, and extracting an interaction feature using the feature of the inspector and the feature of the assessment subject.

Here, the method may further include extracting a context feature from the input video from which information about the inspector and the assessment subject is removed.

Here, the interaction feature may be extracted by inputting the feature of the inspector, the feature of the assessment subject, and the context feature.

Here, the method may further include outputting an interaction-based ASD diagnostic aid result based on the interaction feature.

Here, the method may further include outputting a subject-action-based ASD diagnostic aid result using the feature of the assessment subject.

Here, a feature corresponding to each of the inspector and the assessment subject may include a gaze feature, a facial expression feature, an action feature, and a voice feature.

Here, extracting the feature of the inspector and the feature of the assessment subject may comprise generating a multimodal feature by fusing the gaze feature, the facial expression feature, the action feature, and the voice feature.

Here, extracting the detection area and voice corresponding to the assessment subject from the input video may comprise extracting the detection area and the voice using previously input image and voice information of the assessment subject.

Here, the feature of the inspector may be generated through an inspector feature extraction network by inputting the detection area and voice corresponding to the inspector thereto, and the feature of the assessment subject may be generated through a subject feature extraction network by inputting the detection area and voice corresponding to the assessment subject thereto.

Also, in order to accomplish the above objects, an apparatus for supporting ASD diagnosis based on AI according to an embodiment of the present disclosure includes an inspector extraction unit for extracting a detection area and voice corresponding to an inspector from an input video, a subject extraction unit for extracting a detection area and voice corresponding to an assessment subject from the input video, an individual feature extraction unit for extracting a feature of the inspector and a feature of the assessment subject, and an interaction feature extraction unit for extracting an interaction feature using the feature of the inspector and the feature of the assessment subject.

Here, the apparatus may further include a context feature extraction unit for extracting a context feature from the input video from which information about the inspector and the assessment subject is removed.

Here, the interaction feature may be extracted by inputting the feature of the inspector, the feature of the assessment subject, and the context feature.

Here, the apparatus may further include a diagnostic aid result output unit for outputting an interaction-based ASD diagnostic aid result based on the interaction feature.

Here, the diagnostic aid result output unit may output a subject-action-based ASD diagnostic aid result using the feature of the assessment subject.

Here, a feature corresponding to each of the inspector and the assessment subject may include a gaze feature, a facial expression feature, an action feature, and a voice feature.

Here, the individual feature extraction unit may generate a multimodal feature by fusing the gaze feature, the facial expression feature, the action feature, and the voice feature.

Here, the subject extraction unit may extract the detection area and the voice using previously input image and voice information of the assessment subject.

Here, the feature of the inspector may be generated through an inspector feature extraction network by inputting the detection area and voice corresponding to the inspector thereto, and the feature of the assessment subject may be generated through a subject feature extraction network by inputting the detection area and voice corresponding to the assessment subject thereto.

### BRIEF DESCRIPTION OF THE DRAWINGS

The above and other objects, features, and advantages of the present disclosure will be more clearly understood from the following detailed description taken in conjunction with the accompanying drawings, in which:

FIG. 1 is a flowchart illustrating a method for supporting ASD diagnosis based on AI according to an embodiment of the present disclosure;

FIG. 2 is a configuration diagram for a method for supporting ASD diagnosis based on AI according to an embodiment of the present disclosure;

FIG. 3 is a view illustrating in detail the configuration of an inspector feature extractor;

FIG. 4 is a view illustrating in detail the configuration of a subject feature extractor;

FIG. 5 is a view illustrating in detail the configuration of a context information and interaction feature extractor;

FIG. 6 is a block diagram illustrating an apparatus for supporting ASD diagnosis based on AI according to an embodiment of the present disclosure; and

FIG. 7 is a view illustrating the configuration of a computer system according to an embodiment.

### DESCRIPTION OF THE PREFERRED EMBODIMENTS

The advantages and features of the present disclosure and methods of achieving them will be apparent from the following exemplary embodiments to be described in more detail with reference to the accompanying drawings. However, it should be noted that the present disclosure is not limited to the following exemplary embodiments, and may be implemented in various forms. Accordingly, the exemplary embodiments are provided only to disclose the present disclosure and to let those skilled in the art know the category of the present disclosure, and the present disclosure is to be defined based only on the claims. The same reference numerals or the same reference designators denote the same elements throughout the specification.

It will be understood that, although the terms “first,” “second,” etc. may be used herein to describe various elements, these elements are not intended to be limited by these terms. These terms are only used to distinguish one element from another element. For example, a first element discussed below could be referred to as a second element without departing from the technical spirit of the present disclosure.

The terms used herein are for the purpose of describing particular embodiments only and are not intended to limit the present disclosure. As used herein, the singular forms are intended to include the plural forms as well, unless the context clearly indicates otherwise. It will be further understood that the terms “comprises,” “comprising,”, “includes” and/or “including,” when used herein, specify the presence of stated features, integers, steps, operations, elements, and/or components, but do not preclude the presence or addition of one or more other features, integers, steps, operations, elements, components, and/or groups thereof.

In the present specification, each of expressions such as “A or B”, “at least one of A and B”, “at least one of A or B”, “A, B, or C”, “at least one of A, B, and C”, and “at least one of A, B, or C” may include any one of the items listed in the expression or all possible combinations thereof.

Unless differently defined, all terms used herein, including technical or scientific terms, have the same meanings as terms generally understood by those skilled in the art to which the present disclosure pertains. Terms identical to those defined in generally used dictionaries should be interpreted as having meanings identical to contextual meanings of the related art, and are not to be interpreted as having ideal or excessively formal meanings unless they are definitively defined in the present specification.

Hereinafter, embodiments of the present disclosure will be described in detail with reference to the accompanying drawings. In the following description of the present disclosure, the same reference numerals are used to designate the same or similar elements throughout the drawings, and repeated descriptions of the same components will be omitted.

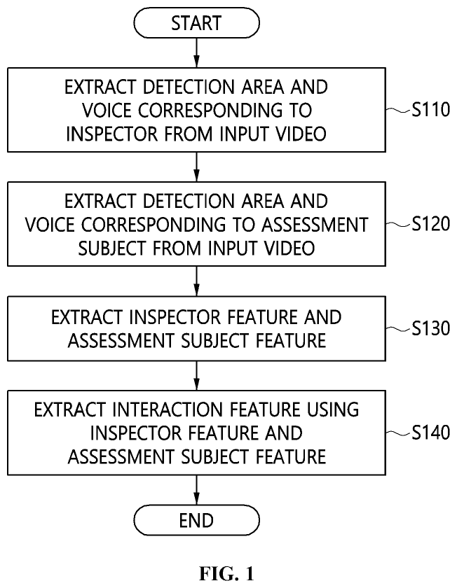

FIG. 1 is a flowchart illustrating a method for supporting ASD diagnosis based on AI according to an embodiment of the present disclosure.

The method for supporting ASD diagnosis based on AI according to an embodiment of the present disclosure may be performed by an apparatus for supporting ASD diagnosis, such as a computing device, a server, or the like. Here, the method according to an embodiment of the present disclosure may be performed by a single device or server, but may be alternatively performed by multiple devices.

Referring to FIG. 1, the method for supporting ASD diagnosis based on AI according to an embodiment of the present disclosure includes extracting a detection area and voice corresponding to an inspector from an input video at step S110, extracting a detection area and voice corresponding to an assessment subject from the input video at step S120, extracting the feature of the inspector and the feature of the assessment subject at step S130, and extracting an interaction feature using the feature of the inspector and the feature of the assessment subject at step S140.

Here, the method may further include extracting a context feature from the input video from which information about the inspector and the assessment subject is removed.

Here, the interaction feature may be extracted by inputting the feature of the inspector, the feature of the assessment subject, and the context feature.

Here, the method may further include outputting an interaction-based ASD diagnostic aid result based on the interaction feature.

Here, the method may further include outputting a subject-action-based ASD diagnostic aid result using the feature of the assessment subject.

Here, a feature corresponding to each of the inspector and the assessment subject may include a gaze feature, a facial expression feature, an action feature, and a voice feature.

Here, extracting the feature of the inspector and the feature of the assessment subject at step S130 may comprise generating a multimodal feature by fusing the gaze feature, the facial expression feature, the action feature, and the voice feature.

Here, extracting the detection area and voice corresponding to the assessment subject from the input video at step S120 may comprise extracting the detection area and the voice using previously input image and voice information of the assessment subject.

Here, the feature of the inspector may be generated through an inspector feature extraction network by inputting the detection area and voice corresponding to the inspector thereto, and the feature of the assessment subject may be generated through a subject feature extraction network by inputting the detection area and voice corresponding to the assessment subject thereto.

A key indicator in ASD diagnosis is to perform evaluation based on observation of stimulus/response behavior appearing in social interaction with others and behavior of repeating the same action regardless of external stimuli, such as stereotyped behavior. The present disclosure presents a Deep Neural Network architecture capable of classifying ASD based on the behavior of social interaction between two people and effectively evaluating behavior unrelated to external stimuli.

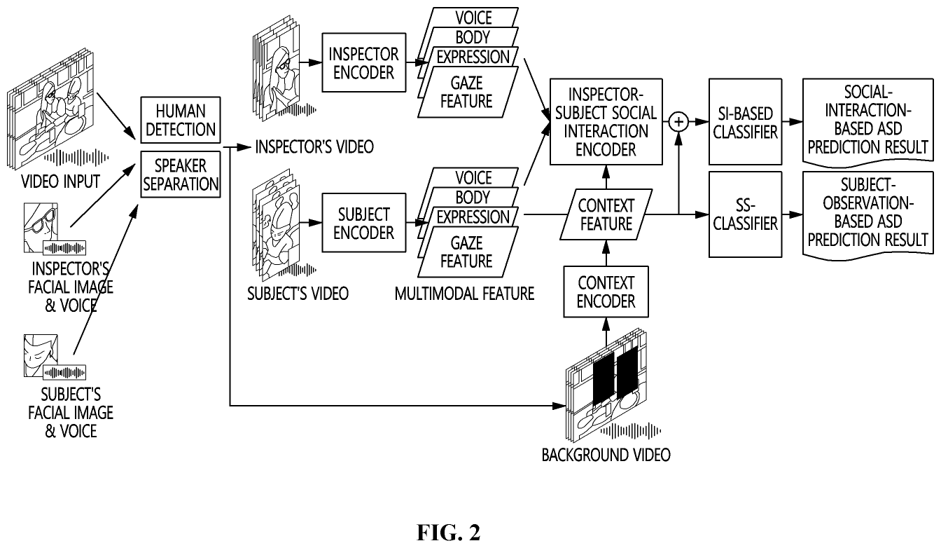

FIG. 2 is a configuration diagram for a method for supporting ASD diagnosis based on AI according to an embodiment of the present disclosure.

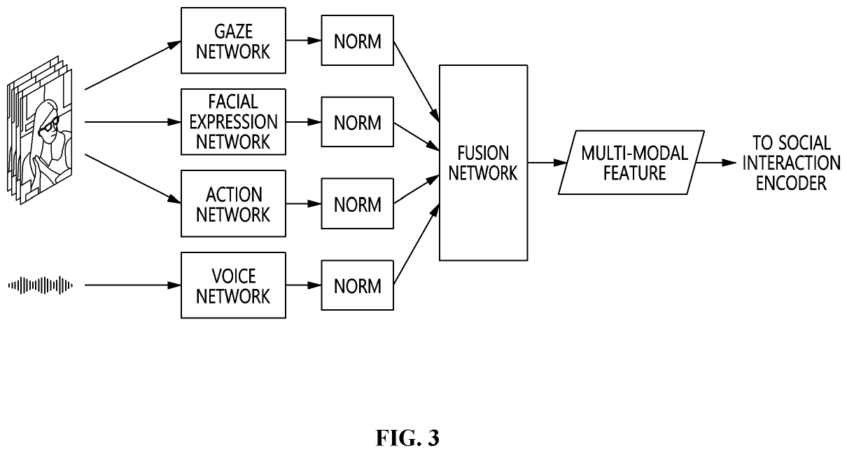

FIG. 3 is a view illustrating in detail the configuration of an inspector feature extractor.

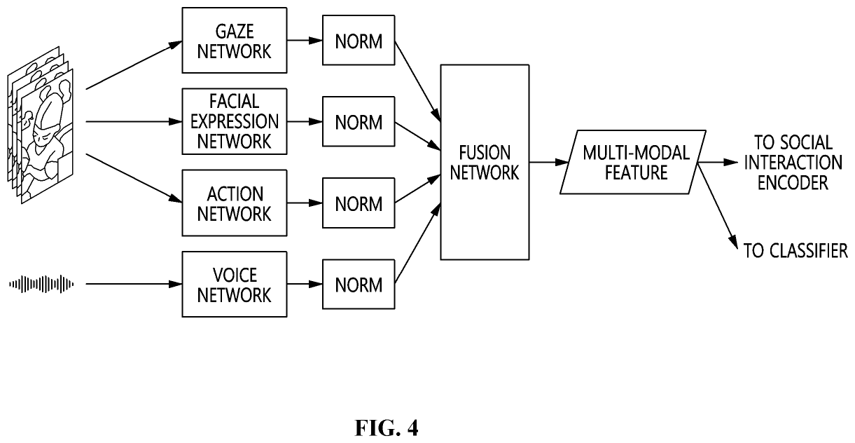

FIG. 4 is a view illustrating in detail the configuration of a subject feature extractor.

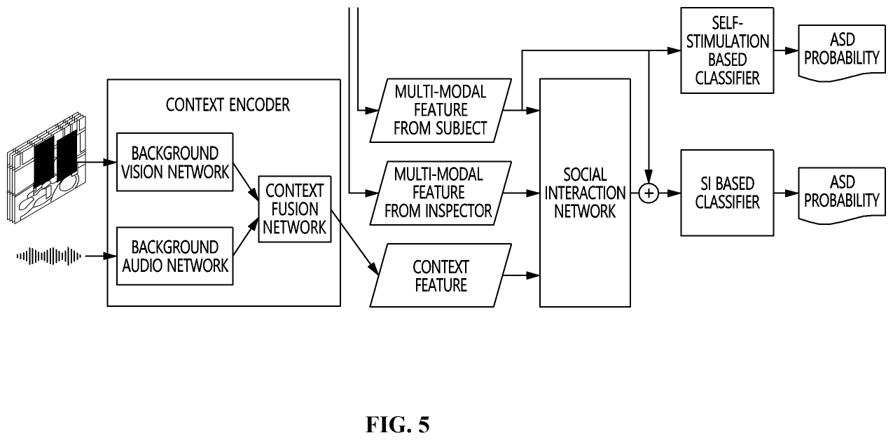

FIG. 5 is a view illustrating in detail the configuration of a context information and interaction feature extractor.

In the method for supporting ASD diagnosis based on AI according to an embodiment of the present disclosure, facial images and voices of an inspector and an assessment subject may be received such that the inspector and the assessment subject can be identified.

Here, the assessment subject may be a subject for whom ASD screening or diagnosis is required, and the inspector may be a person who performs social interaction with the assessment subject. For human detection and speaker separation, the facial images and voices of the inspector and those of the assessment subject are input, and the identification and voice of each of the inspector and the assessment subject are recognized through the human detection and speaker separation, whereby human detection areas and separated voices of the inspector and assessment subject may be obtained. The human detection and speaker separation are preprocessing for automatic ASD diagnosis, and this preprocessing may be manually performed. Pretraining may be performed for the human detection and speaker separation.

After the human area and voice information of the inspector are separated from those of the assessment subject, the human area and voice information of the inspector and those of the assessment subject may be respectively input to an inspector feature extractor (inspector encoder) and a subject feature extractor (subject encoder). Extracting the feature of the inspector and extracting the feature of the assessment subject are performed separately, whereby stimulus and response behavior in the social interaction may be comprehensively understood.

FIG. 3 and FIG. 4 illustrate the detailed configuration diagrams of multimodal feature extractors of an inspector and an assessment subject. The features of the inspector and the assessment subject may be extracted as multimodal features, such as gaze, a facial expression, an action (body), voice, and the like, which are main traits of ASD. Here, it is assumed that a network for extraction of each modality feature is pretrained. Because the feature of the inspector is distinguished from the feature of the assessment subject, when pretraining is performed, training can be performed using data generalized from the features.

For example, when the assessment subject is an infant/child, a multimodal feature extractor may be trained using a dataset for infants/children. Also, because the inspector typically performs actions for eliciting responses, a dataset satisfying such characteristics may be used to train the multimodal feature extractor. Using the pretrained multimodal feature extractor, the weights of the inspector/subject feature extractors are initialized, and when training is performed later, the difference between the inspector and the assessment subject may be represented by performing fine-tuning.

The extracted feature of the inspector and that of the assessment subject may be respectively input to a social interaction feature extractor (social interaction encoder). The respectively input features are used to extract a complicated and integrated interaction feature in the social interaction feature extractor. Social interaction feature extraction requires integrated analysis of complex stimulus/response behavior of two people.

For example, whether stimulus/response behaviors occur chronologically or concurrently may be an important indicator for ASD diagnosis. Also, information about whether the gaze is directed at the other person when social smiling emerges is an important criterion for determination. In consideration of complexity/integration of such social interaction, a separate social interaction feature extractor is proposed.

Context information excluding two people communicating with each other in social interaction may also be an important ASD diagnosis indicator. The context information may include all information that can affect social interaction behavior, such as background information, audio information, and the like. In the present disclosure, explicit feature extraction for the context information is performed. That is, a video, excluding the inspector area and the assessment-subject area, and audio information after speaker separation are input to a context feature extractor (context encoder), and features are extracted therefrom and then included as the main features in the social interaction. Accordingly, the social interaction feature extractor may analyze all of the context feature and the features of the inspector and assessment subject. FIG. 5 illustrates the detailed configuration diagram of the social interaction feature extractor and the context feature extractor.

The present disclosure provides a skip connection for extracting a multimodal feature of an assessment subject and directly inputting the same to a classifier for ASD classification, separately from extraction of a social interaction feature, thereby providing a structure capable of reflecting behavior features unrelated to external stimuli. Because special behavior unrelated to external stimuli (stereotyped behavior or the like), which is one of key diagnostic indicators of ASD, is not associated with social interaction, ASD diagnosis may be more accurately performed through such a design point. In FIG. 4, the design is made such that the multimodal feature is directly provided to the classifier.

The main characteristics of the present disclosure are as follows. First, in order to analyze nonverbal interaction behavior of each of the inspector and the assessment subject, the individual multimodal features may be extracted.

Also, in order to perform complex analysis of social interaction behavior between the inspector and the assessment subject, extraction of social interaction features based on individual multimodal feature extraction may be performed.

Also, a skip connection for analyzing behavior unrelated to the social interaction with others, that is, stereotyped behavior, may be configured.

Also, explicit context information feature extraction may be performed using information (e.g., background/situation information), excluding information about the inspector and the assessment subject.

The present disclosure uses an RGB camera and a microphone as input devices, and may operate in computers to which video and audio information can be input. The algorithm proposed in the present disclosure enables efficient training and inference using GPU hardware capable of parallel processing. In the present disclosure, a deep neural network may be used as a feature extractor (encoder) for extracting key features of video and audio, but other algorithms in a learnable form may be used.

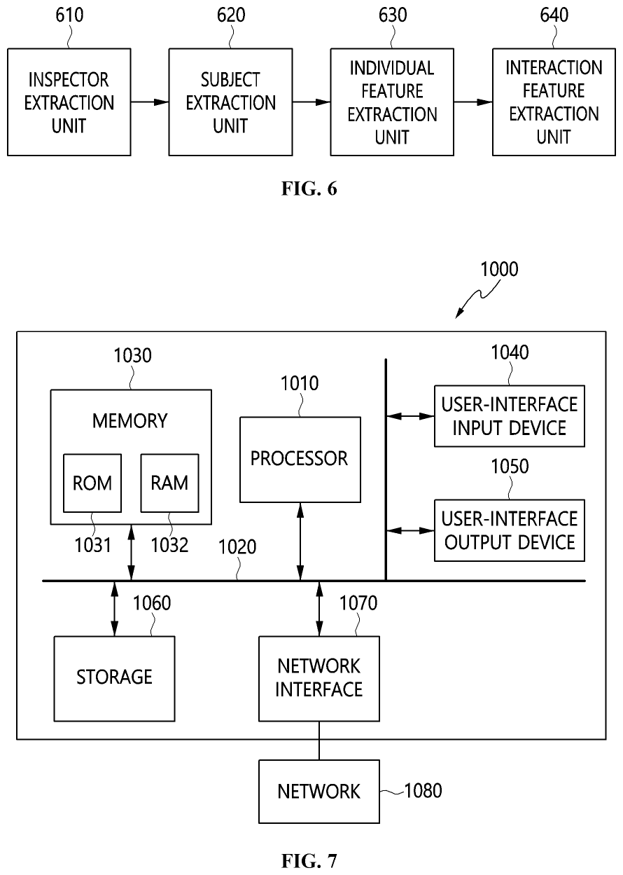

FIG. 6 is a block diagram illustrating an apparatus for supporting ASD diagnosis based on AI according to an embodiment of the present disclosure.

Referring to FIG. 6, the apparatus for supporting ASD diagnosis based on AI according to an embodiment of the present disclosure includes an inspector extraction unit for extracting a detection area and voice corresponding to an inspector from an input video, a subject extraction unit for extracting a detection area and voice corresponding to an assessment subject from the input video, and an individual feature extraction unit for extracting a feature of the inspector and a feature of the assessment subject, and an interaction feature extraction unit for extracting an interaction feature using the feature of the inspector and the feature of the assessment subject.

Here, the apparatus may further include a context feature extraction unit for extracting a context feature from the input video from which information about the inspector and the assessment subject is removed.

Here, the interaction feature may be extracted by inputting the feature of the inspector, the feature of the assessment subject, and the context feature.

Here, the apparatus may further include a diagnostic aid result output unit for outputting an interaction-based ASD diagnostic aid result based on the interaction feature.

Here, the diagnostic aid result output unit may output a subject-action-based ASD diagnostic aid result using the feature of the assessment subject.

Here, the feature corresponding to each of the inspector and the assessment subject may include a gaze feature, a facial expression feature, an action feature, and a voice feature.

Here, the individual feature extraction unit may generate a multimodal feature by fusing the gaze feature, the facial expression feature, the action feature, and the voice feature.

Here, the subject extraction unit may extract the detection area and the voice using previously input image and voice information of the assessment subject.

Here, the feature of the inspector may be generated through an inspector feature extraction network by inputting the detection area and voice corresponding to the inspector thereto, and the feature of the assessment subject may be generated through a subject feature extraction network by inputting the detection area and voice corresponding to the assessment subject thereto.

FIG. 7 is a view illustrating the configuration of a computer system according to an embodiment.

The apparatus for supporting ASD diagnosis based on AI according to an embodiment may be implemented in a computer system 1000 including a computer-readable recording medium.

The computer system 1000 may include one or more processors 1010, memory 1030, a user-interface input device 1040, a user-interface output device 1050, and storage 1060, which communicate with each other via a bus 1020. Also, the computer system 1000 may further include a network interface 1070 connected to a network 1080. The processor 1010 may be a central processing unit or a semiconductor device for executing a program or processing instructions stored in the memory 1030 or the storage 1060. The memory 1030 and the storage 1060 may be storage media including at least one of a volatile medium, a nonvolatile medium, a detachable medium, a non-detachable medium, a communication medium, or an information delivery medium, or a combination thereof. For example, the memory 1030 may include ROM 1031 or RAM 1032.

According to the present disclosure, the interaction behavior between an inspector and an assessment subject is analyzed, whereby ASD diagnosis may be supported.

Also, the present disclosure may support ASD diagnosis by analyzing social context information.

Specific implementations described in the present disclosure are embodiments and are not intended to limit the scope of the present disclosure. For conciseness of the specification, descriptions of conventional electronic components, control systems, software, and other functional aspects thereof may be omitted. Also, lines connecting components or connecting members illustrated in the drawings show functional connections and/or physical or circuit connections, and may be represented as various functional connections, physical connections, or circuit connections that are capable of replacing or being added to an actual device. Also, unless specific terms, such as “essential”, “important”, or the like, are used, the corresponding components may not be absolutely necessary.

Accordingly, the spirit of the present disclosure should not be construed as being limited to the above-described embodiments, and the entire scope of the appended claims and their equivalents should be understood as defining the scope and spirit of the present disclosure.

What is claimed is:

1. A method for supporting Autism Spectrum Disorder (ASD) diagnosis based on Artificial Intelligence (AI), performed by an apparatus for supporting ASD diagnosis, comprising:
extracting a detection area and voice corresponding to an inspector from an input video;
extracting a detection area and voice corresponding to an assessment subject from the input video;
extracting a feature of the inspector and a feature of the assessment subject; and
extracting an interaction feature using the feature of the inspector and the feature of the assessment subject.

2. The method of claim 1, further comprising:
extracting a context feature from the input video from which information about the inspector and the assessment subject is removed.

3. The method of claim 2, wherein the interaction feature is extracted by inputting the feature of the inspector, the feature of the assessment subject, and the context feature.

4. The method of claim 3, further comprising:
outputting an interaction-based ASD diagnostic aid result based on the interaction feature.

5. The method of claim 4, further comprising:
outputting a subject-action-based ASD diagnostic aid result using the feature of the assessment subject.

6. The method of claim 1, wherein a feature corresponding to each of the inspector and the assessment subject includes a gaze feature, a facial expression feature, an action feature, and a voice feature.

7. The method of claim 6, wherein extracting the feature of the inspector and the feature of the assessment subject comprises generating a multimodal feature by fusing the gaze feature, the facial expression feature, the action feature, and the voice feature.

8. The method of claim 1, wherein extracting the detection area and voice corresponding to the assessment subject from the input video comprises extracting the detection area and the voice using previously input image and voice information of the assessment subject.

9. The method of claim 1, wherein:
the feature of the inspector is generated through an inspector feature extraction network by inputting the detection area and voice corresponding to the inspector, and
the feature of the assessment subject is generated through a subject feature extraction network by inputting the detection area and voice corresponding to the assessment subject.

10. An apparatus for supporting Autism Spectrum Disorder (ASD) diagnosis based on Artificial Intelligence (AI), comprising:
an inspector extraction unit for extracting a detection area and voice corresponding to an inspector from an input video;
a subject extraction unit for extracting a detection area and voice corresponding to an assessment subject from the input video;
an individual feature extraction unit for extracting a feature of the inspector and a feature of the assessment subject; and
an interaction feature extraction unit for extracting an interaction feature using the feature of the inspector and the feature of the assessment subject.

11. The apparatus of claim 10, further comprising:
a context feature extraction unit for extracting a context feature from the input video from which information about the inspector and the assessment subject is removed.

12. The apparatus of claim 11, wherein the interaction feature is extracted by inputting the feature of the inspector, the feature of the assessment subject, and the context feature.

13. The apparatus of claim 12, further comprising:
a diagnostic aid result output unit for outputting an interaction-based ASD diagnostic aid result based on the interaction feature.

14. The apparatus of claim 13, wherein the diagnostic aid result output unit outputs a subject-action-based ASD diagnostic aid result using the feature of the assessment subject.

15. The apparatus of claim 10, wherein a feature corresponding to each of the inspector and the assessment subject includes a gaze feature, a facial expression feature, an action feature, and a voice feature.

16. The apparatus of claim 15, wherein the individual feature extraction unit generates a multimodal feature by fusing the gaze feature, the facial expression feature, the action feature, and the voice feature.

17. The apparatus of claim 10, wherein the subject extraction unit extracts the detection area and the voice using previously input image and voice information of the assessment subject.

18. The apparatus of claim 10, wherein:
the feature of the inspector is generated through an inspector feature extraction network by inputting the detection area and voice corresponding to the inspector, and
the feature of the assessment subject is generated through a subject feature extraction network by inputting the detection area and voice corresponding to the assessment subject.
